(19) United States
(12) Patent Application Publication
(10) Pub. No.: US 2004/0243042 A1
(43) Pub. Date: Dec. 2, 2004

(54) Facial masks for managing skin wounds

(76) Inventor: Roger D. A. Lipman, Turnhout (BE)

Correspondence Address: Heidi A. Boehlefeld, Renner, Otto, Boisselle & Sklar, LLP, 1621 Euclid Avenue, Nineteenth Floor, Cleveland, OH, 44115-2191, US

(21) Appl. No.: 10/848,641

(22) Filed: May 19, 2004

Related U.S. Application Data
(60) Provisional application No. 60/472,224, filed on May 20, 2003.

(51) Int. Cl. A61F 13/00; A61F 15/00

(52) U.S. Cl. 602/43

(57) ABSTRACT

This invention relates to a facial mask comprising an covering layer having a first and second surface being cut into a mask shape and having a fluid absorbent adhesive on its first surface. In one embodiment, the covering layer is an occlusive layer. The mask may further include an adjunct moisture absorbing layer between the adhesive layer and the first surface of the covering layer. In another embodiment, the mask further comprises a compressibility layer between the adhesive layer and the first surface of the covering layer. The mask may also include a moldable fluid absorbing layer on the surface of the fluid absorbing adhesive opposite the covering layer. The masks of the present invention promote healing of skin wounds, especially the skin wounds form cosmetic procedures such as laser reconstruction.

This application claims priority to provisional application Serial No. 60/472,224 filed on May 20, 2003, the content of which is hereby incorporated by reference in its entirety.

### TECHNICAL FIELD

The present invention relates to facial masks for use in wound management, e.g., on mammalian skin surfaces. In particular, the present invention relates to a mask for use in, e.g., skin resurfacing procedures.

### BACKGROUND

Skin resurfacing techniques include use of lasers, dermabrasion and chemical peels with alpha-hydroxy acids, phenol or trichloroacetic acid. These techniques, and particularly laser techniques, require extensive follow-up treatment to achieve the desired goal of skin resurfacing. The incidences of cosmetic surgery are increasing. It is believed that at least 300,000 such procedures are carried out each year in North America and Europe.

Despite the large numbers of such procedures, there remains a need for materials, methods and systems for treating and dressing wounds such as laser wounds. It is desirable to accelerate the healing and to restore the functional barrier property of the skin as rapidly as possible. Skin resurfacing procedures result in predictable post-operative sequelae including facial edema, wound exudate and erythema. In addition, there may be pain, pruritis, hyperpigmentation, milia formation and acne. It is desirable to address all of these consequences of wounding and aspects of healing. Thus, dressings used should absorb wound exudate sufficiently to minimize dressing changes, and should minimize incidence of wound infection, as well as reducing pain and pruritis, as well as contributing to a cosmetically acceptable result.

Pressure-sensitive adhesive hydrocolloid formulations are comprised of dispersions of fluid absorbent materials in pressure-sensitive adhesive matrices. Many such adhesives are known in medical fields where they find use as functional components of medical devices. In particular, hydrocolloid adhesives have been extensively utilized in the fields of ostomy care and wound care. In ostomy care, hydrocolloid adhesives are used as the barrier adhesive to hold the pouch in place on the peristomal skin, and to protect this skin from the excoriating effects of body waste. In wound care, hydrocolloid adhesives have been used as the basis of absorbent dressings for direct application to especially chronic wounds.

Hydrocolloid pressure-sensitive adhesive compositions are sometimes not very flexible or conformable, so that adhesion to movable and curved body parts often is difficult. This lack of flexibility can give rise to problems. For example, an ostomy patient with folded or scarred skin in the region of his stoma may have difficulty in adhering the hydrocolloid adhesive barrier of his pouch to the peristomal skin without getting a leak between the skin and the adhesive. Leaks would allow highly irritating and excoriating feces or urine to come into contact with the skin. In order to aid leak free adhesion of barriers to undulating skin surfaces, paste products are sometimes used against the skin to fill depressions caused for example by scar tissue or by obesity.

One of the elements needed for such treatment is a mask capable of use on and adherence to, contoured, uneven surfaces such as a human face, particularly in the nasal area. For these reasons, a need remains for a pressure-sensitive adhesive hydrocolloid composition that is moldable, dispensable from a tube, free of solvents, not overly sticky and yet remains moldably adherent to contoured surfaces such as the human face.

### SUMMARY

This invention relates to a facial mask for wound management comprising a conformable covering layer having a first and second surface and being cut into a mask shape; and a fluid absorbent adhesive layer having a first surface and a second surface wherein the first surface of the covering layer overlies the second surface of the adhesive layer. In one embodiment, the covering layer is an occlusive layer. The mask may further include an adjunct moisture absorbing layer between the adhesive layer and the first surface of the covering layer. The mask may further include a compressibility layer between the adhesive layer and the first surface of the covering layer. In another embodiment, the mask further comprises a moldable fluid absorbing layer on the surface of the fluid absorbing adhesive opposite the covering layer.

The fluid absorbing adhesive layer may comprise at least one adhesive and at least one water soluble and/or water swellable polymer. In one embodiment, the adhesive layer comprises at least one thermoplastic elastomer; at least one liquid rubber; at least one polyisobutylene; and at least one oil.

The masks of the present invention promote healing of skin wounds, especially the skin wounds from cosmetic procedures such as laser reconstruction.

### DRAWINGS

FIG. 1 is a schematic cross-sectional view of a mask in accordance with an embodiment of the present invention.

FIG. 2 is a schematic cross-sectional view of a mask having a moldable fluid absorbing layer in accordance with another embodiment of the present invention.

FIG. 3 is a schematic cross-sectional view of a mask with an adjunct absorbing layer in accordance with another embodiment of the present invention.

FIG. 4 includes a schematic cross-sectional view of a mask with an adjunct absorbing layer and a moldable fluid absorbing layer in accordance with another embodiment of the present invention.

FIG. 5 is a schematic depiction of addressing in the form of a mask, in accordance with an embodiment of the present invention.

It should be appreciated that for simplicity and clarity of illustration, elements shown in the Figures have not necessarily been drawn to scale. For example, the dimensions of some of the elements may be exaggerated relative to each other for clarity. Further, where considered appropriate, reference numerals have been repeated among the Figures to indicate corresponding elements.

### DETAILED DESCRIPTION

The masks may form coverings for a portion or all of a patient's face. Such a mask may have one or more prefabricated openings, corresponding to eyes, nasal openings and/or mouth. In one embodiment, the mask is split into sub-facial parts, either latitudinally, longitudinally, both latitudinally and longitudinally, or diagonally. As described herein, the facial masks help promote skin healing. The masks have a covering layer and one or more fluid absorbing layers. The masks may have one or more intermediate layers. The intermediate layers are between the covering layer and the fluid absorbing adhesive layer. The intermediate layers may provide increased fluid absorption, improved cushioning or improved conformation to the face.

As used herein, a mask may include, as well as a mask with openings appropriate for use on the face of a human patient, a mask with openings, separations and/or score lines appropriate for use on other body parts of a human patient, and a mask with openings, separations and/or score lines appropriate for use with non-human living beings. The latter masks may be for veterinary use, for example, in treating injured animals. Thus, a mask may include a dressing that is appropriately shaped, provided with openings, separations and/or score lines for application to various body parts of living beings as needed and as appropriate to the skin of the particular living being which is being treated.

As described herein, the mask comprises a covering layer and a fluid absorbing adhesive layer. The covering layer overlays the fluids absorbing adhesive layer directly or through one or more intermediate adhesive layer.

Covering Layer

The covering layer may be any material typically used in medical applications. In one embodiment, the covering is any suitable polymeric film, plastic foam (including open celled foam), a woven fabric, knitted fabric or a non-woven fabric. The fabric and non-woven materials may be those typically used in bandages. The covering is sometimes referred to as a top layer.

In one embodiment, the occlusive top layer comprises a material which is generally impervious to fluid transmission, but which allows for some degree of moisture vapor transmission. In one embodiment, the occlusive top layer is a material which substantially prevents transmission of liquid water, but which allows transmission of water vapor. The occlusive top layer, in one embodiment, has a moisture vapor transmission rate (MVTR) of substantially zero. In another embodiment, the occlusive top layer has a moisture vapor transmission rate (MVTR) in the range of about 100 to 2000 g/m²/24 hours. In one embodiment, from about 400 to about 1000 g/m²/24 hours, and in one embodiment from about 600 to about 800 g/m²/24 hours. In one embodiment, where larger dressings are needed and treatment requires that a portion of the wound fluid be evaporated through the dressing, the MVTR may be as high as 4000 g/m²/24 hours or greater. In one embodiment, the occlusive top layer allows some degree of moisture vapor transmission, but is still occlusive overall in that it prevents a significant drying of the dressing and the underlying skin wound.

In one embodiment, the occlusive top layer is flexible yet resistant to tearing. In one embodiment, the thickness of the occlusive top layer is from about 4 micrometers (μm) to about 1500 μm (1.5 mm). In another embodiment, the thickness of the occlusive top layer is from about 15 μm to about 500 μm. In another embodiment, the thickness of the occlusive top layer is from about 20 μm to about 250 μm. In yet another embodiment, the thickness of the occlusive top layer is from about 25 μm to about 125 μm.

The occlusive top layer may be opaque or translucent. In one embodiment, it has a skin color, but colors and patterns may be used. The occlusive top layer may be solid or porous, permeable or perforated, as adapted for the needs of the patient, as well as being a function of the composition and form of the occlusive top layer material.

In one embodiment, the occlusive top layer is substantially impervious to liquid, especially wound exudate. In yet another embodiment, the occlusive top layer is substantially impervious to bacteria. In another embodiment, the occlusive top layer is capable of absorbing liquid, especially wound exudate. In another embodiment, perforations or small apertures in the occlusive top layer facilitate a high rate moisture vapor transmission.

In one embodiment, the occlusive top layer may comprise any suitable polymeric film, plastic foam (including open celled foam), a woven fabric, knitted fabric or a non-woven fabric. In one embodiment, the fabrics may be natural or synthetic materials. In one embodiment, the occlusive top layer possesses at least some moisture vapor transmission capability, which may also be referred to as breathability.

In one embodiment, the occlusive top layer comprises a film comprised of a polymeric material including, for example, polyurethanes, polyolefins such as linear low density polyethylene, low density polyethylene and ethylene vinyl acetate, Saran materials such as vinylidene chloride copolymers of vinyl chloride, methyl acrylate, or methyl methacrylate copolymers. In one embodiment, the polymeric material is polyurethane, either as a film or as a polyurethane foam. In one embodiment, the polyurethane may be an ester or ether based polyurethane, such as MEDIFILM®. In other embodiments, materials that can be used in the occlusive top layer are styrene copolymers such as styrene-butadiene-styrene (S-B-S), styrene-isoprene-styrene (S-I-S) and styrene-ethylene/butylenes-styrene (S-EB-S), methyl methacrylate copolymers, polyethylene copolymers and nitrile rubber. In other embodiments, the occlusive top layer may comprise a moisture vapor permeable film produced from synthetic polymers which are capable of being formed into continuous films by casting, extrusion or other known film-making processes.

In one embodiment, the occlusive top layer may comprise, for example, thermoplastic polyurethanes such as Dow Chemical Company's PELLETHANE®, including its 2363-80AE grade thereof; K. J. Quinn's Q-THANE®; B. F. Goodrich's ESTANE®; Mobay Chemical Company's TXIN®; and others. In other embodiments, the occlusive top layer can also comprise various polyesters, such as the copolymers of various cyclic polyesters including DuPont's HYTREL®, including its 4056 grade thereof, and General Electric's LOMOD®, both of which are copolymers of polyether prepolymers and polybutylene terephthalate and polyisobutyl terephthalate, respectively, as well as Eastman Chemical's PCCE®. PCCE® is a copolymer of polyether and polyethylene terephthalate, such as PCCE®9965 from Eastman Chemical Products, Inc., Other suitable flexible copolyesters are PCCE® 9966 and PCCE® 9967 also available from Eastman. These other suitable copolyesters are characterized by the use of 1,4 cyclohexane dimethanol, 1,4 cyclohexane dicarboxylic acid, and polytetramethylene glycol ether as reactants in producing the flexible copolyester resins.

Also useful for the occlusive layer is ethylene methyl acrylate (EMA). Suitable EMAs may comprise from about 10 to about 20 wt. % methyl acrylate. EMAs are easily stretchable and conformable, and have very low, and in one embodiment, substantially zero, MVTR. In one embodiment, the occlusive layer is EMA and has an MVTR in the ranges described above. A suitable EMA is LOTRYL®24MA005 (EMA) available from AtoChem, with a tensile strength of 2910 psi, elongation of 700%, a-melt index of 0.5, a Durometer of 84 A, a melting point of 70° C., and a Vicat Softening index of 43.

Also suitable are AtoChem LOTRYL® olefin acrylic copolymer grades 17BA01, 17BG04,17BA04, 17BA07, 28BA175, 30BA02, 35BA40, 35BA320 (which are Ethyl Butyl Acrylate (EBA) copolymers), and 9MA02, 14MG02, 15MA03, 16MA03, 18MA02, 18MG02, 20MA08, 24MA005, 28MA07, 28MA175, 29MA03, and 35MA05 (which are Ethyl Methyl Acrylate (EMA) copolymers).

Exxon OPTEMA® TC221 EMA is another suitable EMA.

Fluid Absorbing Adhesive

The fluid absorbing adhesive layer comprises (a) at least one adhesive and (b) at least one water-soluble and/or water-swellable polymer. The fluid absorbing adhesive is, in one embodiment, non-adherent to the wound. In another embodiment, the fluid absorbing adhesive layer is moldable. The adhesive portion of the layer is generally present in an amount from about 10% to about 80%, or from about 15% to about 70%, or from about 20% to about 60% by weight of the fluid absorbing layer. The adhesive portion comprises an adhesive and optionally one or more other additives. The water soluble and/or water swellable polymers are generally present in an amount from about 20% to about 90%, 30% to about 85%, or from about 40% to about 80% by weight of the fluidizing layer. Here and elsewhere the specification and claims the range and ratio limits may be combined.

Useful rubber-based pressure sensitive adhesives (PSAs) include those taught in U.S. Pat. No. 5,705,551 (Sasaki et al.) and in U.S. Pat. No. 4,080,348 (Korpman), the disclosures of which are hereby incorporated by reference. Examples of polymeric rubber bases include one or more of styrene-isoprene-styrene polymers, styrene-olefin-styrene polymers including styrene-ethylene/propylene-styrene polymers, polyisobutylene, styrene-butadiene-styrene polymers, polyisoprene, polybutadiene, natural rubber, silicone rubber, acrylonitrile rubber, nitrile rubber, polyurethane rubber, polyisobutylene rubber, butyl rubber, halobutyl rubber including bromobutyl rubber, butadiene-acrylonitrile rubber, polychloroprene, and styrene-butadiene rubber.

The adhesive material of the fluid-absorbing adhesive may include a variety of pressure-sensitive adhesive materials known in the art. In one embodiment, the adhesive may include any medical grade adhesive. The medical adhesives include suitable acrylic based PSAs, suitable rubber-based pressure sensitive adhesives and suitable silicone pressure sensitive adhesives.

In one embodiment, the adhesive material may include solid rubbers such as linear or radial A-B-A block copolymers or mixtures of these A-B-A block copolymers with simple A-B block copolymers. In one embodiment, the proportion of A-B block copolymers, relative to the A-B-A block copolymers, are less than 85% by weight of the (total) block copolymers. In one embodiment, the proportion is in the range from about 35 to about 85% by weight of the block copolymers, and in another embodiment, the proportion is from about 55 to about 75% by weight of the block copolymers. In one embodiment, lower amounts such as 10 to 35% by weight of the block copolymers are used. These block copolymers can be based on styrene-butadiene, styrene-isoprene, and hydrogenated styrene-diene copolymers such as styrene ethylene-butylene.

Suitable styrene-diene copolymers are exemplified by a blend of linear styrene-isoprene-styrene triblock copolymer and linear styrene-isoprene diblock copolymer. Such a material is available from Kraton Polymers as KRATON® D-1161K and has a bound styrene content of about 15% and a diblock content of 17%. A second example is a blend of linear styrene-isoprene-styrene triblock copolymer and linear styrene-isoprene diblock copolymer available from Shell Chemical as KRATON® D-1117 and which has a bound styrene content of about 17% and a diblock content of 33%.

An example of a suitable hydrogenated styrene-diene copolymer is a thermoplastic elastomer comprising a blend of clear linear triblock and diblock copolymer based on styrene and ethylene-butylene with a bound styrene of 14% mass. Such a material is commercially available from Shell Chemical Company as KRATON® G-1657. Another example is KRATON® G-1652 from Shell Chemical Company, which is a thermoplastic elastomer comprised of a clear linear triblock copolymer based on styrene and ethylene-butylene, S-E/B-S, with a bound styrene content of about 30% by weight. Also suitable are polymers in which there is a combination of chemically saturated blocks and chemically unsaturated blocks. For example, a branched copolymer consisting of two polyisoprene chains attached to the rubber midblock of a styrene/ethylene-butylene/styrene triblock copolymer. Such a material is available from Shell Chemical Company as KRATON® Research Product RP6919. This material has a styrene content of 18%, and isoprene content of 36% and an ethylene-butylene content of 46% by weight. Also, a low styrene synthetic copolymer of butadiene and styrene, commonly called SBR rubber, can be used as a solid rubber.

In one embodiment, the adhesive material includes physically cross-linked domains or areas. In another embodiment, the adhesive material is free of physically cross-linked domains or areas. As used herein, “physically cross-linked” means that the crosslinks in the polymer of which it is comprised are not of a chemical (covalent or ionic) nature but of a physical nature. This means that there are areas or domains within the elastomer that have a high crystallinity, i.e., a high glass transition temperature. The term “physically crosslinked” may also be referred to as “pseudo cross-linked”. For example, in a styrene-isoprene block copolymer, the polystyrene regions tend to associate into glassy islands or domains. As known in the art, a polymer may include either or both of physical and chemical (covalent or ionic) crosslinks. Thus, in some embodiments of the present invention, the polymers of the adhesive material may comprise either or both of physical or chemical crosslinks. In one embodiment, only physical crosslinks are present. In another embodiment, only chemical crosslinks are present.

In one embodiment, a particularly useful rubber-based adhesive comprises an adhesive having a thermoplastic elastomeric component and a resin component. The thermoplastic elastomeric component contains about 55-85 parts of a simple A-B block copolymer wherein the A-blocks are derived from styrene homologs and the B-blocks are derived from isoprene, and about 15-45 parts of a linear or radical A-B-A block copolymer wherein the A-blocks are derived from styrene or styrene homologs and the B-blocks are derived from conjugated dienes or lower alkenes, the A-blocks in the A-B block copolymer constituting about 10-18 percent by weight of the A-B copolymer and the total A-B and A-B-A copolymers containing about 20 percent or less styrene.

In one embodiment, the adhesive component includes a styrene-containing thermoplastic elastomer and a liquid rubber. In one embodiment, the styrene-containing thermoplastic elastomer comprises a block or radial copolymer and a liquid rubber. In one embodiment, the permanently tacky pressure-sensitive adhesive is present in a range from about 1 wt. % to about 15 wt. %, or from about 1 wt. % to about 10 wt. % of the adhesive composition.

Suitable styrene-containing permanently tacky pressure-sensitive adhesives useful in this embodiment include a styrenic component and a liquid rubber component. In one embodiment, use of the liquid rubber component with the styrenic component obviates the need for a conventional tackifier.

The styrenic components may include block or radial copolymers based on styrene-butadiene, styrene-isoprene or styrene ethylene-butylene. In addition, a low styrene synthetic copolymer of butadiene and styrene, commonly called SBR rubber, can be used as the thermoplastic elastomer. The elastomer may comprise linear or radial A-B-A block copolymers or mixtures of these A-B-A copolymers with simple A-B block copolymers. In one embodiment, the proportion of A-B block copolymers in the mixture of A-B-A and A-B block copolymers does not exceed about 85% by weight, and in other embodiments, lower percentages are used. In one embodiment, the proportion of A-B block copolymers in the mixture of A-B-A and A-B block copolymers does not exceed about 65% by weight, in another embodiment the proportion of A-B block copolymers in the mixture of A-B-A and A-B block copolymers does not exceed about 50% by weight, and in another embodiment, the proportion of A-B block copolymers in the mixture of A-B-A and A-B block copolymers does not exceed about 35% by weight, and in yet another embodiment, the proportion of A-B block copolymers in the mixture of A-B-A and A-B block copolymers does not exceed about 20% by weight. Here and elsewhere in the specification and claims, the limits of the ranges and ratios may be combined.

In one embodiment, the elastomeric component comprises linear or radial A-B-A block copolymers or mixtures of these linear or radial A-B-A block copolymers with simple A-B block copolymers. In these block copolymers the A-blocks are derived from styrene or styrene homologs and the B-blocks are derived from conjugated dienes or lower alkenes.

The A-B-A block copolymers are of the type which consist of A blocks derived from styrene or one of its homologs and B blocks derived from conjugated dienes, such as butadiene or isoprene, or from lower alkenes such as ethylene or butylene. The radial A-B-A polymers useful in this embodiment are of the type described for example in U.S. Pat. No. 3,281,383 and conform to the general formula (A-B)ₙX, where A and B comprise blocks derived from the monomers described above in connection with the A-B-A copolymers, X is an organic or inorganic connecting moiety having a functionality of at least 2, and n is equal to the functionality of X. Homologs of styrene may include any known homolog of styrene for use in such A-B-A, A-B or (A-B)ₙX copolymers. For example, (C₁-C₈) alkylstyrenes such as α-methylstyrene, o-, m- and p-methylstyrenes, p-ethylstyrene, 2,4-dimethylstyrene, p-n-butylstyrene, p-t-butylstyrene, p-n-hexylstyrene, p-n-octylstyrene, p-n-nonylstyrene and p-n-decylstyrene), arylstyrenes (such as p-phenylstyrene), alkoxy-substituted styrenes (such as p-methoxystyrene), hydroxyl-substituted styrenes (such as p-hydroxystyrene), halogen-substituted styrenes (such as p-chlorostyrene and 3,4-dichlorostyrene) and mixtures of two or more of these (such as mixtures of styrene with at least one substituted styrenes) may be suitable styrene homolog. As used in the following, “styrene” may include homologs thereof.

Suitable styrene-diene copolymers are exemplified by a blend of linear styrene-isoprene-styrene triblock copolymer and linear styrene-isoprene diblock copolymer. Such a material is available from Kraton Polymers, Houston, Tex. as KRATON® D-1161K (referred to as D-1161 in Europe) and has a bound styrene content of about 15% and a diblock content of 17%. A second example is a blend of linear styrene-isoprene-styrene triblock copolymer and linear styrene-isoprene diblock copolymer available from Shell Chemical as KRATON® D-1117P and which has a bound styrene content of about 17% and a diblock content of 33%.

An example of a suitable hydrogenated styrene-diene copolymer is a thermoplastic elastomer comprising a blend of clear linear triblock and diblock copolymer based on styrene and ethylene-butylene with a bound styrene of about 14 wt. %. Such a material is commercially available from Kraton Polymers as KRATON® G-1657M, which has a bound styrene content of about 13 wt. %. Another example is KRATON® G-1 652 (referred to as G-1652E in Europe) from Kraton Polymers, which is a thermoplastic elastomer comprised of a clear linear triblock copolymer based on styrene and ethylene-butylene, S-E/B-S, with a bound styrene content of about 30 wt. %. Also suitable are polymers in which there is a combination of chemically saturated blocks and chemically unsaturated blocks. For example, a branched copolymer consisting of two polyisoprene chains attached to the rubber midblock of a styrene/ethylene-butylene/styrene triblock copolymer. Such a material is available from Kraton Polymers as KRATON® Research Product RP6919. This material has a styrene content of 18 wt. %, an isoprene content of 36 wt. % and an ethylene-butylene content of 46 wt. %.

Useful acrylic based PSAs include those taught in U.S. Pat. No. 5,947,917 (Carte), and U.S. Pat. No. 5,164,444 (Bernard, acrylic emulsion), U.S. Pat. No. 5,623,011 (Bernard, tackified acrylic emulsion). The acrylic PSA may also comprise a radiation curable mixture of monomers with initiators and other ingredients such as those taught in U.S. Pat. No. 5,232,958 (Ang, UV cured acrylic) and U.S. Pat. No. 5,232,958 (Mallya et al, EB cured). The disclosures of these patents as they relate to acrylic adhesives are hereby incorporated by reference.

In one embodiment, the adhesive material includes a silicone-based pressure-sensitive adhesive. Useful silicone pressure sensitive adhesives include those commercially available from Dow Corning Corp., Medical Products and those available from General Electric. Examples of silicone adhesives available from Dow Corning include those sold under the trade names BIO-PSA X7-3027, BIO-PSA X7-4919, BIO-PSA X7-2685, BIO-PSA X7-3122 and BIO-PSA X7-4502. Additional examples of silicone pressure sensitive adhesives useful in the present invention are described in U.S. Pat. Nos. 4,591,622, 4,584,355, 4,585,836 and 4,655,767.

A processing stabilizer of the IRGANOX® 1010 described below may be used to protect the thermoplastic elastomer from excessive degradation during processing.

The fluid absorbing adhesive may include one or more optional ingredients such as liquid rubbers, low molecular weight polyisobutylene, high molecular weight polyisobutylene, etc. These materials are each present in an amount from about 1% to about 35%, or from about 2% to about 25%, or from about 3% to about 15% by weight of the adhesive composition.

The adhesive composition of the present invention includes, in one embodiment, an adhesive component including a styrene-containing thermoplastic elastomer; a liquid rubber; a polyisobutylene; and an oil. This adhesive component is combined or mixed with the at least one water-soluble and/or water-swellable absorbent polymer to form a moldable pressure-sensitive adhesive hydrocolloid composition. In another embodiment, the adhesive composition of the present invention includes a permanently tacky pressure-sensitive adhesive component comprising a styrene-containing block copolymer and a liquid rubber; a low molecular weight polyisobutylene; and mineral oil.

In another embodiment, a moldable composition comprises a continuous phase consisting of a mixture of a permanently tacky pressure sensitive adhesive, preferably a hot melt adhesive based on a styrene-containing thermoplastic elastomer and a liquid rubber, a low molecular weight polyisobutylene and a low molecular weight liquid polybutene, and dispersed within the continuous phase a discontinuous phase of one or more water soluble and/or water swellable absorbent polymers.

Liquid rubbers are synthetic liquid isoprene rubber, depolymerised natural rubber, carboxyl terminated synthetic liquid isoprene-styrene rubber, hydroxyl terminated synthetic liquid isoprene rubber, hydrogenated liquid isoprene rubber, liquid isoprene-styrene copolymer, liquid isoprene-butadiene copolymer and liquid butadiene-styrene copolymer. In one embodiment, the liquid rubbers have a molecular weight in a range from about 2500 to about 50,000. In one embodiment, the liquid rubbers have a glass transition temperature of less than about −50° C., and a melt viscosity at 38° C. in the range from about 500 to about 10,000 poises. It will be appreciated that other liquid rubbers known in the art could be useful in this embodiment of the present invention. The liquid rubbers are generally present in an amount from about 1% to about 25%, or from about 2% to about 15%, or from about 3% to about 10% by weight of the adhesive composition.

In one embodiment, the thermoplastic elastomer comprises a block copolymer of styrene and isoprene having a styrene content of about 13 wt. % and an isoprene content of about 87 wt. %, a glass transition temperature of about −60° C., a melt viscosity of about 2400 poises at 50° C. and has a weight average molecular weight of about 30,000 to about 50,000. KRATON® LVSI-101 is a material having such properties.

Another example of a useful liquid rubber is a liquid polyisoprene obtained by selectively or partially degrading a high molecular weight polyisoprene. An example of a commercially available partially degraded high molecular weight polyisoprene is ISOLENE® D-400 from Elementis Performance Polymers, Belleville, N.J., and this liquid rubber has a molecular weight of about 20,000. Other liquid rubbers that may be incorporated into the adhesive mixture include liquid styrene-butadiene rubbers, liquid butadiene rubbers, ethylene-propylene rubbers, etc., as noted above.

In one embodiment, the liquid rubber component comprises a low molecular weight liquid rubber, as defined below. In one embodiment, the liquid rubber component has a number average molecular weight less than about 3,000.

A block copolymer of styrene and isoprene having a styrene content of about 13% and an isoprene content of about 87%, a glass transition of about −60° C., a melt viscosity of about 240 Pas at 50° C. and which is commercially available from Shell Chemical Company as LVSI101, is particularly useful in the practice of the invention. Within the adhesive material, in one embodiment, the weight ratio of solid rubber to liquid rubber is in the range from about 100:1 to about 1:2, and is varied in order to obtain the desired degree of adhesiveness and tackiness. In one embodiment, the weight ratio of solid rubber to liquid rubber is in the range from about 50:1 to about 5:1, and in another embodiment, from about 20:1 to about 10:1.

In one embodiment, the adhesive comprises a weight ratio of styrene-containing block copolymer to liquid rubber of about 1:0.25 to about 1:10, and in one embodiment from about 1:0.5 to about 1:7, and in another embodiment, from about 1:1.5 to about 1:3.5.

In one embodiment, the amount of liquid rubber is present in an amount from 0.5% to about 15%, or from about 1% to about 10%, or form about 2% to about 7% of the total formulation.

In one embodiment, the adhesive composition further includes a polyisobutylene component. In one embodiment, the polyisobutylene component is a low molecular weight polyisobutylene. In one embodiment, the polyisobutylene component is exemplified by the VISTANEX® LM series of polyisobutylenes, available from ExxonMobil Chemical Corporation. In one embodiment, the polyisobutylene has a Flory viscosity average molecular weight in the range from about 25,000 to about 75,000, and in one embodiment, from about 35,000 to about 70,000, and in one from about 40,000 to about 55,000. In one embodiment, the polyisobutylene has a Brookfield viscosity at 1 75° C. within the range from about 10,000 to about 170,000 mpa.sec, and in one embodiment, from about 20,000 to about 140,000 mpa.sec, and in another from about 25,000 to about 70,000 mpa.sec. Brookfield viscosity is determined by measuring the shearing stress on a spindle rotating at a definite, constant speed while it is immersed in the sample. Brookfield viscosity is measured in centipoises or mPa.sec. Viscosity is a function of shear rate and is defined as shear stress/shear rate, and is measured according to ASTM D3236. In one embodiment, the Staudinger molecular weight of the low molecular weight polyisobutylenes ranges from about 5,000 to about 20,000, and in another embodiment, the Staudinger molecular weight ranges from about 10,000 to about 12,000.

The optional low molecular weight polybutene components are exemplified by the HYVIS® series of materials from BP, and by the PARAPOL® series of products from Exxon Chemical Corporation, and which have molecular weights in the range from about 1000 to about 3000, determined using test method AM-I 841-86, and kinematic viscosities at 100° C. within the range from about 180 to about 3500 cSt, as measured by test method ASTM D445.

In one embodiment, the low molecular weight polyisobutylene component is present in the moldable pressure-sensitive adhesive hydrocolloid composition at between about 5% to about 50%, or at between 25 wt. % and about 45 wt. % of the total formulation, and in one embodiment, between about 30 wt. % and about 40 wt. % of the total formulation.

In one embodiment, the adhesive composition further includes an oil component. In one embodiment, the oil component comprises mineral oil. In some embodiments, a portion of the mineral oil may be replaced by the liquid hydrocarbon or liquid polymeric material or by a natural vegetable oil. In one embodiment, the mineral oil is exemplified by the Kaydol series of materials from Witco Chemical. Witco White Mineral Oil USP has a viscosity at 40° C. between about 60 and about 75 mm²/s, and in one embodiment, between about 63 and about 70 mm²/s, as determined by test method ASTM D-445. However, any suitable mineral oil may be used. Mineral oil may also be referred to as liquid petrolatum, mineral spirits, adepsine oil, alboline, glymol, liquid paraffin, paraffin oil or saxol, some of which may be used as trade names. Mineral oil has a boiling point in the range from about 179° C. to about 210° C.

In one embodiment, rather than mineral oil, the oil component may comprise corn oil, soybean oil, cottonseed oil, castor bean oil, palm oil, coconut oil, sunflower seed oil, canola oil, other known vegetable oils, animal oils such as fish oil, lard and tallow, and may further comprise synthetic oils triglycerides.

In one embodiment, the oil component is present in the moldable pressure-sensitive adhesive hydrocolloid composition at between about 5% to about 60%, or between about 25 wt. % and about 45 wt. %, and in one embodiment, from about 30 wt. % to about 40 wt.

In one embodiment a portion of the oil component may be replaced by a liquid hydrocarbon or a liquid polymeric component, as defined above. In one such embodiment, at least about 25 wt. % of the composition is mineral oil, and the additional liquid hydrocarbon or liquid polymer component is added thereto, up to a total limit of about 45 wt. % for the combined mineral oil and liquid hydrocarbon or liquid polymeric component.

In one embodiment, a suitable processing stabilizer may also be included in the moldable pressure-sensitive adhesive hydrocolloid composition. Suitable stabilizers include those indicated for use with styrene-olefin-styrene block copolymer thermoplastic elastomers such as organophosphites and the so-called hindered phenols, but any suitable stabilizers may be employed. An example of an organophosphite stabilizer is tris(nonylphenyl)phosphite, available as POLYGARD® HR, manufactured by Uniroyal.

Particularly useful stabilizers are the hindered phenols, IRGANOX® 1010 and IRGANOX® 565, manufactured by Ciba. IRGANOX® 1010 is pentaerythritol tetrakis (3-(3,5-di-tert-butyl-4hydroxyphenyl) propionate. IRGANOX® 565 is 2,6-di-tert-butyl4-(4, 6-bis (octylthio)-1,3,5-triazin-2yl-amino)phenol. Stabilizers may be used separately or in combination, and suitable ranges are within about 0.1% to about 1.5%, or in one embodiment, from about 0.3% to about 1%, by weight based on the total formulation. When present, the stabilizers are added to the adhesive component, as is shown in the examples.

Other optional ingredients such as tackifiers and plasticisers may be added to the adhesive composition, to modify tack and optimize adhesion properties. However, tackifiers used in prior art hydrocolloid adhesives may make the present composition too sticky. Thus, in one embodiment, the moldable pressure-sensitive adhesive hydrocolloid composition is substantially free of any added tackifier. As used herein, the term “tackifier” or “tackifying resin” includes: (a) natural and modified rosins such, for example, as gum rosin, wood rosin, tall-oil rosin, distilled rosin, hydrogenated rosin, dimerized rosin, and polymerized rosin; (b) glycerol and pentaerythritol esters of natural and modified rosins, such, for example, as in the gycerol ester of pale wood rosin, the glycerol ester of hydrogenated rosin, the glycerol ester of polymerized rosin, the pentaerythritol ester of hydrogenated rosin, and the phenolic modified pentaerythritol ester of rosin; polyterpene resins having a softening point, as determined by ASTM method E28 58T, of from about 60° C. to 140° C., the latter polyterpene resins generally resulting from the polymerization of terpene hydrocarbons, such as the bicyclic mono-terpene known as pinene, in the presence of Friedel-Crafts catalysts at moderately low temperatures; (d) phenolic-modified terpene resins such, for example, as the resin product resulting from the condensation in an acidic medium, of a bicyclic terpene and a phenol; and (e) aliphatic petroleum hydrocarbon resins having a Ball and Ring softening point of from about 60° C. to 140° C., the latter resins resulting from the polymerization of monomers consisting primarily of olefins and diolefins. Thus, the low molecular weight polyisobutylenes disclosed for use herein are not included within the definition of tackifier as used herein. In addition, tackifying resins are usually solids at ordinary temperatures, while the low molecular weight polyisobutylenes disclosed herein are liquids at ordinary temperatures Optional fillers such as silica and pigments and optional active ingredients such as antimicrobial compounds may also be incorporated into the moldable pressure-sensitive adhesive hydrocolloid compositions of this embodiment of the invention. Silver sulfadiazine and benzalkonium chloride represent non-limiting examples of such antimicrobial ingredients.

In one embodiment, the moldable pressure-sensitive adhesive hydrocolloid composition is substantially free of added wax, mineral wax or petroleum jelly, and in one embodiment is substantially free of microcrystalline wax. In one embodiment, the moldable pressure-sensitive adhesive hydrocolloid composition is substantially free of copolymers such as ethylene vinyl acetate, and in one embodiment the composition is substantially free of copolymers of ethylene vinyl alcohol. In one embodiment, the moldable pressure-sensitive adhesive hydrocolloid composition is substantially free of additives such as aloe, aloe vera extract, etc. In one embodiment, the moldable pressure-sensitive adhesive hydrocolloid composition is substantially free of absorbent additives such as silica, aerosil, diatomaceous earth, zeolites or molecular sieve. In one embodiment, the moldable pressure-sensitive adhesive hydrocolloid composition is substantially free of solvent. In one embodiment, the moldable pressure-sensitive adhesive hydrocolloid composition is substantially resin-free. In one embodiment, the moldable pressure-sensitive adhesive hydrocolloid composition is substantially free of a tackifier. In one embodiment, the moldable pressure-sensitive adhesive hydrocolloid composition is substantially free of radiation cross-linked polymers.

Water-Soluble and/or Water-Swellable Polymer

The fluid absorbing adhesive layer also comprises at least one water-soluble and/or water-swellable polymer. In one embodiment, the polymer is a hydrophilic polymer that is soluble or insoluble but swellable in water as the moisture-absorbing component. At least one swellable polymer may be present in the composition. Suitable insoluble swellable polymers include cross-linked sodium carboxymethyl cellulose, crystalline sodium carboxymethyl cellulose, cross-linked dextran and starch-acrylonitrile graft copolymer. The swellable polymer may also include a so-called “super absorbent” material such as starch sodium polyacrylate. Other hydratable polymers such as gluten and polymers of methyl vinyl ether and maleic acid and derivatives thereof may also be included in the water-soluble and/or water-swellable polymer. Suitable water soluble polymers include sodium carboxymethyl cellulose, pectin, gelatine, guar gum, locust bean gum, collagen, karaya gum and starch, particularly maize starch, and the like.

In one embodiment, the hydrophilic absorbent polymers comprise at least one of alginic acid, sodium alginate, calcium alginate, cellulose-derived material, starch or a modified starch, a copolymer of a starch or a cellulosic material, a water soluble hydrocolloid, a synthetic resin, a mannan, seaweeds, a plant mucilage.

In one embodiment, the water-soluble and/or water-swellable polymer is about 60% or less of the total weight of the moldable pressure-sensitive adhesive hydrocolloid composition, in one embodiment, the water-soluble and/or water-swellable polymer constitutes about 35 to about 55% by weight of the moldable pressure-sensitive adhesive hydrocolloid composition, and in another embodiment, the water-soluble and/or water-swellable polymer constitutes from about 40 to about 50% by weight of the moldable pressure-sensitive adhesive hydrocolloid composition.

The water-soluble and/or water-swellable polymer may be comprised of any combination of soluble and/or insoluble absorbents. The swellable polymer may also be a so-called “super absorbent” material such as starch sodium polyacrylate. In one embodiment, the swellable polymer is other than a “super absorbent” material. Other hydrophilic absorbent polymers such as gluten and polymers of methyl vinyl ether and maleic acid and derivatives thereof may also be included in the water-soluble and/or water-swellable polymer.

In one embodiment, the water-soluble and/or water-swellable polymer may comprise at least one water-soluble hydrocolloid, alone or blended with at least one swellable polymer. Such soluble hydrocolloids include naturally derived products such as pectin, gelatin, starches, guar gum, locust bean gum, gum arabic, gum karaya, collagen, karaya gum, alginic acid and its sodium and/or calcium salts. Also useful are the synthetic hydrocolloids such as sodium carboxymethyl cellulose, crosslinked sodium carboxymethyl cellulose, polyvinyl alcohol, polyvinyl pyrrolidone, high molecular weight polyethylene glycols and polypropylene glycols.

In one embodiment, the water-soluble and/or water-swellable polymer may comprise at least one of, e.g., starches such as flour starch, corn starch, potato starch, etc. In another embodiment, mannan such as yeast gum, manna or konjak. In another embodiment, the water-soluble and/or water-swellable polymer may comprise at least one of various seaweeds such as agar-agar, sodium alginate, etc. In another embodiment, the water-soluble and/or water-swellable polymer may comprise at least one plant mucilage such as tragacanth gum, gum arabic, karaya gum, guar gum, psyllium seed gum, dammar gum, pectin etc., various proteins such as gelatin, collagen, casein, etc. In another embodiment, the water-soluble and/or water-swellable polymer may comprise at least one cellulose-derived material such as carboxymethyl cellulose, hydroxyethyl cellulose, methyl cellulose, etc., modified starches such as soluble starch, carboxymethyl starch, dialdehyde starch, a cross-linked dextrin, etc. In another embodiment, the water-soluble and/or water-swellable polymer may comprise at least one copolymer of starch or cellulose, such as starch-acrylonitrile graft copolymer, a starch polyacrylate salt. In another embodiment, the water-soluble and/or water-swellable polymer may comprise at least one synthetic resin such as polyvinyl alcohol, sodium polyacrylate, polyethylene oxide, etc., and copolymers of starches or celluloses and acrylonitrile, acrylic acid, methacrylic acid, vinyl alcohol, vinyl chloride, etc. In some embodiment, the water-soluble and/or water-swellable polymer may comprise at least one of plant mucilage such as tragacanth gum, gum arabic, karaya gum, guar gum, psyllium seed gum, dammar gum, pectin, etc., the celluloses such as CMC (carboxymethyl cellulose), HEC (hydroxyethl cellulose), etc., and the copolymers of starches or celluloses and acrylonitrile, acrylic acid, sulfuric acid, vinyl sulfonate, etc. The foregoing embodiments may be combined with one another, and may be combined with water swellable polymers and/or super-absorbent materials.

In one embodiment, the water swellable polymers include, for example, hydroxypropylcellulose (HPC) and polyethylene oxide (PEO). HPC is available from commercial suppliers including, for example, Aqualon, Inc., (Wilmington, DE). The useful HPC generally has an average molecular weight in the range of about 60,000 to 1,200,000. In another embodiment, the water swellable polymer includes homopolymers and copolymers of carboxymethyl cellulose, hydroxyethyl cellulose, hydroxymethyl cellulose). In another embodiment, the water swellable polymer includes a water-soluble or water-swellable polymer derived from acrylic acid or a pharmaceutically acceptable salt thereof, such as the polyacrylic acid polymers as follows: Polycarbophil (Noveon AA-1), carbomer (Carbopol 974P or 971P or 907), or a water-soluble salt of a co-polymer of methyl vinyl ether and maleic acid or anhydride (Gantrez MS-955).

Adjunct Moisture Absorbing Layer

In one embodiment, the mask further comprises an adjunct moisture absorbing layer. In one embodiment, the adjunct moisture absorbing layer is a adhesive composition, similar to the above-described, except that it includes a hydrocolloid moisture absorbing material with a very high moisture-absorbing capability as the water-soluble and/or water-swellable polymer. A mask having the adjunct layer is used when the level of exudate is expected to be very high, or when the occlusive dressing must remain in place for an extended length of time. In both situations, the adjunct layer helps to avoid leakage of wound exudate by providing extra capacity to absorb the exudate.

In one embodiment, an adjunct moisture absorbing, e.g., hydrocolloid-containing, layer is laminated between the occlusive layer and the fluid-absorbing pressure-sensitive adhesive layer. This adjunct layer may contain, in one embodiment, an absorbent material capable of absorbing 15 or more grams of physiological saline per gram of absorbent. In one embodiment, the adjunct layer comprises a “super-absorbent” polymer, as described above. Suitable super absorbent materials include the blended nonwoven rayon/“Oasis” superabsorbent fibers available from National Nonwovens, Cincinnati, Ohio. The adjunct layer may include any moldable, putty-like or paste-like hydrocolloid, but in particular may be one of the moldable hydrocolloid adhesive dressings described below.

In one embodiment, the adjunct layer comprises a foam or fibrous structure for absorbing fluids such as wound exudate. In one embodiment, the foam can absorb greater than 250%, in one embodiment at least about 500%, and in another embodiment, at least about 800%, by weight aqueous saline solution based on the dry weight of the foam. These values may be obtained using a saline absorbency test in which a dry, weighed sample is immersed for 30 minutes at 37° C. in phosphate-buffered saline containing 0.9 wt. % NaCl.

In one embodiment, the foam is substantially non-swellable. The term “substantially non-swellable” means that there is little or no increase in volume of the foam upon absorption of water or wound exudate. In one embodiment, the foam increases in volume by no greater than about 10%, and in another embodiment, by no greater than about 5%, when swell tested as above.

Suitable foams may have a wide range of thicknesses. In one embodiment, the foam is at least about 0.5 millimeter, and in another embodiment, at least about 1 millimeter thick. In one embodiment, the foam is no more than about 10-20 millimeters thick.

In one embodiment, the foam is an open-cell foam. Suitable open cell foams may have an average cell size (typically, the longest dimension of a cell, such as the diameter) of at least about 30 microns, and in one embodiment, at least about 50 microns. The cell size, in one embodiment, is no greater than about 800 microns, and in one embodiment, no greater than about 500 microns, as measured by scanning electron microscopy (SEM) or light microscopy. In one embodiment, the foam includes a synthetic polymer that is adapted to form a conformable open cell foam that absorbs the wound exudate. Examples of suitable materials for the absorbent, substantially non-swellable foam include synthetic organic polymers including, but not limited to: polyurethanes, carboxylated butadiene-styrene rubbers, polyesters, and polyacrylates. The polymeric foams can be made of one or more types of monomers (e.g., copolymers) or mixtures (e.g., blends) of polymers. In one embodiment, the foam is a polyurethane, available under the trade designation POLYCRIL® 400 from Fulflex, Inc, Middleton, R.I. In another embodiment, the foam may be a polyester or polyether polyurethane foam which may be formed from either a linear or cross-linked polyurethane. Such foams are known in the art.

In another embodiment, the adjunct layer may comprise an absorbent foam. Such foams are known in the art, and have fluid-absorbing ability similar to the above-described foams.

In one embodiment, the mask includes at least one moisturizing agent. Suitable moisturizing agents include hyaluronic acid, sodium hyaluronate, collagen, corboxymethyldextran, chitosan, squalene and lecithin. Other moisturizing agents known in the art may be used either alone or together with any of the foregoing moisturizing agents.

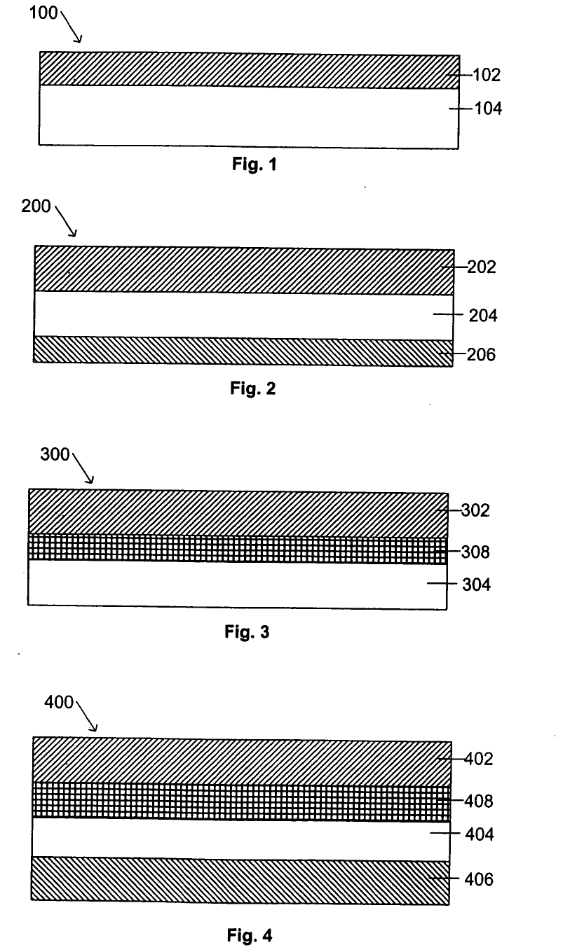

Referring now to the figures, FIG. 1 is a cross section of a mask 100 having a covering 102, such as one of the occlusive films above or a bandage fabric, adhered to a fluid absorbing layer. The fluid absorbing layer is placed against the skin. In one embodiment, the fluid absorbing layer is the moldable fluid absorbing layer described above. In another embodiment, a moldable paste, such as those described above, may be added to the fluid absorbing layer to fill spaces between the face and mask that would have otherwise been bridged or not in contact. For instance, the past may be used in the area of the nose or cheeks to make sure that the face is in contact with the mask through the paste.

FIG. 2 is a cross section of mask 200 having covering layer 202 adhered to fluid adsorbing layer 204. Fluid absorbing layer 204 in turn is adhered to moldable fluid absorbing layer 206.

FIG. 3 is a cross section of mask 300 that has a covering layer 302 adhered to a compressible layer 308. The compressible layer may be a material that is compressible to provide better contact between the face and fluid absorbing layer. Medical foam, such as those described above, moldable fluid absorbing pastes, such as those described above, or a cotton batting may be used as the compressibility layer. In one embodiment, a moldable paste, such as those described above, may be added to the fluid absorbing layer to fill spaces between the face and mask that would have otherwise been bridged or not in contact.

FIG. 4 is a cross section of mask 400 that has a covering 402 which is adhered to compressibility layer 408. Compressibility layer 408 is adhered to fluid absorbing adhesive layer 404. Fluid absorbing adhesive layer 404 is in turn adhered to moldable adhesive layer 406.

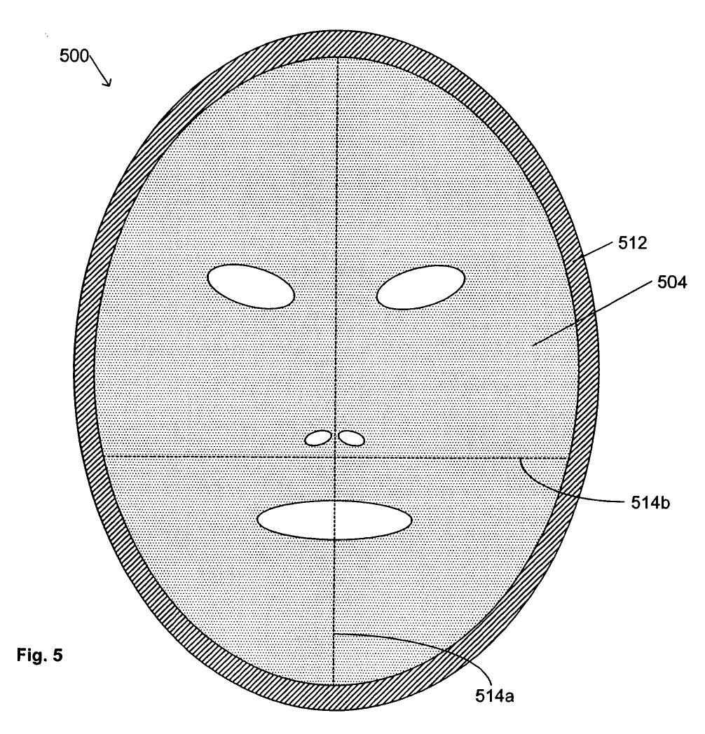

FIG. 5 is a schematic diagram of a dressing in the form of a mask, in accordance with an embodiment of the present invention. As shown in FIG. 5, in one embodiment, a dressing, such as an occlusive, moldable or moisturizing dressing, is provided in the form of a mask 500. The mask 500 includes a dressing 504 and a backing layer 512. Although not shown, the mask may be covered by a release liner, which would be removed prior to use, or may include a carrier layer, which would either be retained or be removed for prior to use. In another embodiment, also not shown, the mask 500 may include an occlusive layer on the upper surface thereof, such as the occlusive layer 102 shown in FIGS. 1 and 2, and described with respect thereto.

As shown in FIG. 5, the mask 500 is provided with openings appropriate for eyes, nasal openings and mouth of a patient. Such an arrangement facilitates use of the mask, by making it more comfortable, easier to apply, easier to remove, and more comprehensively covering the face than other methods, such as strip bandages.

In accordance with the present invention, the mask 500 comprises one of the dressings described above, including both the adhesive material and the water-soluble and/or water-swellable absorbent polymer component. Thus, any of the embodiments described herein may be provided in the form of the mask 500. In one embodiment of the occlusive dressings, the mask forms the occlusive layer. In one embodiment, the mask is provided for combination with any one of the foregoing dressings, the combination to be assembled at the time of use with the patient.

In accordance with another embodiment of the mask 500, the mask may be provided either in separate pieces or with scored lines to enable separation into separate pieces at the time of use. Such an embodiment is schematically shown in FIG. 5 by the dashed lines 514a and 514b. In one embodiment, the mask 500 includes a vertical separation or scored line indicated by the dashed line 514a. In one embodiment, the mask 500 includes a horizontal separation or scored line indicated by the dashed line 514b. In one embodiment, the mask 500 includes both a vertical separation or scored line indicated by the dashed line 514a, and a horizontal separation or scored line indicated by the dashed line 504. It will be recognized that, in accordance with the invention, other separations or scored lines may be included, as needed to provide a mask 500 which is adaptable to the particular needs of physician and patient.

### EXAMPLES

Preparation of Hydrocolloid Compositions:

In one embodiment, the hydrocolloid compositions for use with the masks of this invention may be prepared using a pressure-sensitive adhesive hydrocolloid composition as in the following illustrative, non-limiting examples.

### Example A

A thermoplastic elastomer and a liquid rubber component are blended together in a suitable mixer, normally a sigma blade mixer with an extruder discharge. The mixer is heated to about 170° C. A nitrogen flow of about 60 ml/sec through the mixer reduces the possibility of oxidative degradation of the elastomer during processing. About 1% phr of a suitable stabilizer, for example, IRGANOX® 1010, can be added at this stage. The thermoplastic elastomer is allowed first to blend in the mixer until it coalesces. A small amount of the liquid rubber, 10-20% of the total amount to be added, is added to the whole amount of the thermoplastic elastomer and the liquid rubber is allowed to blend with the softened thermoplastic elastomer. When all this 10-20% of the liquid rubber oil has been absorbed, another portion of the liquid rubber is added, for example about 20-30%, and the liquid rubber is absorbed into the styrene-olefin-styrene rubber. This is continued until all the liquid rubber is added, when a pourable tacky intermediate adhesive is obtained. The mixer blades are stopped, the direction of the screw is reversed, and the intermediate adhesive is removed from the mixer. It is run off into suitably release coated containers and allowed to cool.

The mixer is cleaned, stabilized at 90° C. and the powdery hydrocolloid ingredients are charged to the mixer together with the low molecular weight polyisobutylene and the mineral oil. These are blended until uniform for about 10 minutes and then the previously blended permanently tacky pressure-sensitive adhesive component is added and the mixer temperature is raised to 105° C. After mixing at 105° C. for 15 minutes the temperature of the mixer is dropped to about 80° C. and any other additives, such as the low molecular weight polybutene is added (in various embodiments as appropriate). Mixing is continued normally for a further approximately 20 minutes. The fully mixed mass is then removed from the mixer.

The above manufacturing process has been described with respect to a two-step process in which the permanently tacky pressure-sensitive adhesive comprising the styrene-containing thermoplastic elastomer, liquid rubber and stabilizer is first manufactured and isolated and which is then subsequently used to make a fluid absorbing adhesive composition of this embodiment of the invention. It will be appreciated by anyone of ordinary skill in the art that no process limitation is implied and the adhesives of the invention may equally well be prepared by other methods.

### Example 1

This example illustrates preparation of the intermediate permanently tacky pressure-sensitive adhesive component for use in the mask of the present invention.

| Component | wt. % | Amount, gm |
|---|---|---|
| LVSI-101 | 79.37 | 400 |
| KRATON ® D-1161N | 19.84 | 100 |
| IRGANOX ® 1010 | 0.79 | 4 |
| TOTAL | 100.00 | 504 |

A Z-blade mixer of 1 kg capacity is purged with nitrogen gas and heated to 160° C. The speed of the front, faster, blade is 30 rpm. The KRATON® D-1161 K and the IRGANOX® 1010 are charged to the mixer at 160° C., and the mixer is started. After mixing for 5 minutes, when the rubbery crumb coalesces, 50 gm of the LVSI-101 is added with continued mixing and nitrogen purging. After a further ten minutes, the temperature is raised to 170° C. and the mixer front blade speed increased to 47 rpm. The LVSI had at this point should be completely mixed with the rubber, and a further 51 gm of LVSI is added. Ten minutes later, after blending of the second portion of the LVSI, a further 48 gm of LVSI is added, and is mixed for a further 10 minutes. In this way, approximately 50 gm portions of the charge of LVSI are added every 10 minutes until all the 400 gm has been added. After mixing for another 15 minutes, the intermediate adhesive is dumped from the mixer. The total time for this operation is about 90 minutes.

The above preparation or compounding process has been described with respect to a two-step process in which the hot melt adhesive comprising the styrene-containing thermoplastic elastomer and the liquid rubber and the stabilizer is first manufactured and isolated and which is then subsequently used to make the adhesive compositions of this embodiment of the invention. It will be appreciated by anyone of ordinary skill in the art that no process limitation is implied and the adhesives of the invention may equally well be prepared by other methods.

The foregoing process can be used to prepare the first two ingredients in the following Examples 2-41, which are then subsequently combined with the remaining ingredients as described above.

### Examples 2-41

The following examples illustrate preparations of adhesive hydrocolloid compositions useful in the present invention. In the following examples, the hydrocolloid ingredients, the adhesive and the liquid components are mixed as described above, and the adhesive component, which has been prepared as in Example 1, is combined with these ingredients as described above.

| | 2 | 3 | 4 | 5 | 6 | 7 | 8 | 9 | 10 | 11 |
|---|---|---|---|---|---|---|---|---|---|---|
| Formulation | wt. % | wt. % | wt. % | wt. % | wt. % | wt. % | wt. % | wt. % | wt. % | wt. % |
| LVSI-101 | 7.2 | 7.2 | 2.4 | 4.4 | 1.0 | 1.1 | 5.0 | 6.3 | 3.0 | 4.0 |
| Kraton D-1161 | 1.8 | 1.8 | 0.6 | 1.1 | 0.7 | 0.8 | 1.6 | 1.3 | 0.6 | 1.0 |
| VISTANEX ® LMMS | 20.0 | 23.0 | 23.0 | 23.8 | 25.0 | 27.0 | 19.0 | 29.0 | 30.0 | 23.0 |
| Pectin USP100 | 16.0 | 10.0 | 13.6 | 12.9 | 18.2 | 14.1 | 11.5 | 8.0 | 8.0 | 9.0 |
| Blanose 7H4XF | 16.0 | 14.7 | 13.6 | 12.9 | 7.1 | — | 11.5 | 8.0 | 8.0 | 9.0 |
| GENU Pectin | — | 10.0 | 19.6 | 12.9 | 14.0 | 14.0 | 11.5 | 8.0 | 8.0 | 9.0 |
| LM-104 AS-FS | | | | | | | | | | |
| Calcium Alginate | — | — | — | 7.0 | 14.0 | 7.0 | 11.5 | 15.0 | 8.0 | — |
| Mineral Oil | 39.0 | 33.3 | 30.2 | 25.0 | 20.0 | 32.0 | 32.4 | 24.4 | 34.4 | 45.0 |
| Total | 100.0 | 100.0 | 100.0 | 100.0 | 100.0 | 100.0 | 100.0 | 100.0 | 100.0 | 100.0 |
| | 12 | 13 | 14 | 15 | 16 | 17 | 18 | 19 | 20 | 21 |
| Formulation | wt. % | wt. % | wt. % | wt. % | wt. % | wt. % | wt. % | wt. % | wt. % | wt % |
| Liquid Rubber | 8.0 | 7.5 | 8.0 | 7.5 | 8.0 | 7.5 | 8.0 | 7.5 | 8.0 | 7.5 |
| Poly bd R-45HT | | | | | | | | | | |
| High MW PIB | 1.0 | 1.5 | 1.0 | 1.5 | 1.0 | 1.5 | 1.0 | 1.5 | 1.0 | 1.5 |
| VISTANEX ® | | | | | | | | | | |
| VISTANEX ® LM-MS | 20.0 | 23.0 | 22.0 | 24.0 | 25.0 | 27.0 | 19.0 | 15.0 | 30.0 | 23.0 |
| Pectin USP100 | 16.0 | 10.0 | 11.0 | 13.0 | 18.0 | 14.0 | 10.0 | 10.0 | 6.0 | 7.0 |
| Blanose 7H4XF | 15.0 | 14.0 | 13.0 | 12.0 | 7.0 | — | 10.0 | 9.0 | 6.0 | 9.0 |
| GENU Pectin | — | 9.0 | 15.0 | 10.0 | 14.0 | 11.0 | 10.0 | 9.0 | 7.0 | 7.0 |
| LM-104 AS-FS | | | | | | | | | | |
| Calcium Alginate | — | — | — | 7.0 | 4.5 | 6.5 | 10.0 | 24.0 | 8.0 | — |
| Polyterpene | 1.0 | — | — | — | 0.5 | — | 2.0 | — | — | — |
| Wingtack 95 | | | | | | | | | | |
| Rosin Ester | — | 1.0 | — | — | — | 0.5 | — | 2.0 | — | — |
| Foral 105 | | | | | | | | | | |
| Mineral Oil | 39.0 | 34.0 | 30.0 | 25.0 | 20.0 | 32.0 | 30.0 | 22.0 | 34.0 | 45.0 |
| Total | 100.0 | 100.0 | 100.0 | 100.0 | 100.0 | 100.0 | 100.0 | 100.0 | 100.0 | 100.0 |
| | 22 | 23 | 24 | 25 | 26 | 27 | 28 | 29 | 30 | 31 |
| Formulation | wt % | wt. % | wt. % | wt. % | wt. % | wt. % | wt. % | wt. % | wt. % | wt. % |
| Polyisoprene | 8.0 | 7.5 | 8.0 | 7.5 | 8.0 | 7.5 | 8.0 | 7.5 | 8.0 | 7.5 |
| Kraton IR305 | | | | | | | | | | |
| High MW PIB | 1.0 | 1.5 | 1.0 | 1.5 | 1.0 | 1.5 | 1.0 | 1.5 | 1.0 | 1.5 |
| VISTANEX ® | | | | | | | | | | |
| Polybutene | 20.0 | 23.0 | 22.0 | 24.0 | 25.0 | 27.0 | 19.0 | 15.0 | 30.0 | 23.0 |
| INDOPOL H-300 | | | | | | | | | | |
| Pectin USP100 | 16.0 | 10.0 | 11.0 | 13.0 | 18.0 | 14.0 | 10.0 | 10.0 | 6.0 | 7.0 |
| Blanose 7H4XF | 16.0 | 14.0 | 13.0 | 12.0 | 7.0 | — | 10.0 | 9.0 | 6.0 | 9.0 |
| GENU Pectin | — | 10.0 | 15.0 | 10.0 | 14.0 | 11.0 | 10.0 | 9.0 | 7.0 | 7.0 |
| LM-104 AS-FS | | | | | | | | | | |
| Calcium Alginate | — | — | — | 7.0 | 5.0 | 7.0 | 10.0 | 24.0 | 8.0 | — |
| Mineral Oil | 39.0 | 34.0 | 30.0 | 25.0 | 20.0 | 32.0 | 32.0 | 24.0 | 34.0 | 45.0 |
| Total | 100.0 | 100.0 | 100.0 | 100.0 | 100.0 | 100.0 | 100.0 | 100.0 | 100.0 | 100.0 |
| | 32 | 33 | 34 | 35 | 36 | 37 | 38 | 39 | 40 | 41 |
| Formulation | wt. % | wt. % | wt. % | wt. % | wt. % | wt. % | wt. % | wt. % | wt. % | wt. % |
| High MW PIB | 9.0 | — | 9.0 | — | 9.0 | — | 9.0 | — | — | 23.0 |
| VISTANEX ® | | | | | | | | | | |
| VISTANEX ® LM-MS | 20.0 | 23.0 | 22.0 | 24.0 | 25.0 | — | 19.0 | — | 31.0 | 43.0 |
| Kraton D-1161 | — | 9.0 | — | 9.0 | — | 9.0 | — | 9.0 | 21.0 | — |
| Liquid Rubber | — | — | — | — | — | 27.0 | — | 15.0 | 21.0 | 11.0 |
| Polybd R-45HT | | | | | | | | | | |
| Pectin USP100 | 32.0 | 0 | 11.0 | 13.0 | 18.0 | 14.0 | 30.0 | 10.0 | — | 7.0 |
| Blanose 7H4XF | — | 24.0 | 13.0 | 0 | 7.0 | — | — | 9.0 | 12.0 | 9.0 |
| GENU Pectin | — | 10.0 | 15.0 | 22.0 | 14.0 | 11.0 | — | 9.0 | 7.0 | 7.0 |
| LM-104 AS-FS | | | | | | | | | | |
| Calcium Alginate | — | — | — | 7.0 | 5.0 | 7.0 | 10.0 | 24.0 | 8.0 | — |
| Mineral Oil | 39.0 | 34.0 | 30.0 | 25.0 | 20.0 | 32.0 | 32.0 | 24.0 | 0 | 0 |
| Total | 100.0 | 100.0 | 100.0 | 100.0 | 100.0 | 100.0 | 100.0 | 100.0 | 100.0 | 100.0 |

### MATERIALS USED IN EXAMPLES

Styrenic-based copolymers, such as Kraton D1117.

High molecular weight PIB, such as Vistanex L-140 (mw 2,100,000)

Vistanex L-100 (mw 1,250,000)

Liquid rubber, such as Kraton LVSI

polybutadiene-type APOLY bd R-45HT from Idemitsu Petro-Chemical, Japan

hydrogenated polydiene diol liquid polymer from Shell, L2203, L-1203

Polyisoprene, such as Kraton IR305 form Shell

Natsyn 2210 from Goodyear

Low molecular weight PIB, such as Vistanex LM-MH or -MS (mw 45,000)

Polybutene, such as PARAPOL 1300—from Exxon

HYVIS 30 from BP

Indopol H-100, H-300, H-1500 (H-300 mw =1200)

Mineral Oil Kaydol Mineral Oil B Witco Chemical

Polyterpenes, such as WINGTACK from Goodyear

Rosin esters, such as FORAL 105 from Hercules Chemical

Super Ester W-100 from Arakawa Chemical

Hydrocolloids, such as GENU Pectin USP100 B Hercules Chemical

GENU Pectin LM-104 AS-FS B Hercules Chemical

Blanose 7H4XF B Hercules Chemical

Calcium Alginate B Kelco

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

1. A facial mask for wound management comprising a conformable covering layer having a first and second surface and being cut into a mask shape; and a fluid absorbent adhesive layer having a first surface and a second surface wherein the first surface of the covering layer overlies the second surface of the adhesive layer.

2. The mask of claim 1 wherein the covering layer comprises an occlusive layer.

3. The mask of claim 2 wherein the covering layer has a moisture vapor transmission rate in the range of about 100 to about 2000 g/m²/24 hours.

4. The mask of claim 2 wherein the covering layer has a moisture vapor transmission rate of at least 4000 g/m²/24 hours.

5. The mask of claim 1 wherein the covering layer comprises fabric.

6. The mask of claim 1 wherein the covering layer comprises a polymeric film.

7. The mask of claim 6 wherein the polymeric film is transparent.

8. The mask of claim 1 wherein the covering layer comprises plastic foam.

9. The mask of claim 1 wherein the fluid absorbing adhesive layer comprises at least one adhesive and at least one water soluble and/or water swellable polymer.

10. The mask of claim 9 wherein the adhesive comprises an acrylic based pressure sensitive adhesive, a rubber-based pressure sensitive adhesive, a silicone pressure sensitive adhesive or a combination of two or more thereof.

11. The mask of claim 9 wherein the adhesive comprises at least one medical grade adhesive.

12. The mask of claim 9 wherein the fluid absorbent adhesive comprises sodium carboxymethyl cellulose, dextran, starch-acrylonitirle graft copolymer or a combination of two or more thereof.

13. The mask of claim 9 wherein the fluid absorbent adhesive comprises alginic acid, sodium alginate, calcium alginate, cellulose-derived material, starch or a modified starch, a copolymer of a starch or a cellulosic material, a water soluble hydrocolloid, synthetic resin, mannan, seaweed, plant mucilage or a combination of two or more thereof.

14. The mask of claim 9 wherein the fluid absorbent adhesive comprises pectin, gelatin, starches, guar gum, locust bean gum, gum arabic, collagen, karaya gum, alginic acid and its sodium and calcium salts, sodium carboxymethyl cellulose, polyvinyl alcohol, polyvinyl pyrrolidone, high molecular weight polyethylene glycols and polypropylene glycols or a combination of two or more thereof.

15. The mask of claim 1 wherein the adhesive layer further comprises at least one moisturizing agent.

16. The mask of claim 1 wherein the adhesive layer comprises a thermoplastic polymer and a compatible liquid rubber.

17. The mask of claim 16 wherein the adhesive further comprises one or more low molecular weight polyolefins.

18. The mask of claim 16 wherein the liquid rubber comprises a liquid isoprene polymer, a liquid isoprene-styrene copolymer, a liquid isoprene-butadiene copolymer or combinations of two or more thereof.

19. The mask of claim 1 further comprising an adjunct moisture absorbing layer between the adhesive layer and the first surface of the covering layer.

20. The mask of claim 19 wherein the moisture absorbing layer comprises a hydrocolloid.

21. The mask of claim 19 wherein the moisture absorbing layer comprises a moisture absorbing material capable of absorbing at least 250% by weight saline solution based on the weight of the absorbent material.

22. The mask of claim 19 wherein the moisture absorbing layer comprises a foam.

23. The mask of claim 1 further comprising a compressibility layer between the second surface of the adhesive layer and the first surface of the covering layer.

24. The mask of claim 23 further comprising a fluid absorbing moldable layer on the first surface of the fluid absorbing adhesive layer.

25. The mask of claim 1 further comprising a moldable fluid absorbing layer on the second surface of the fluid absorbing adhesive layer.

26. The mask of claim 1 wherein the adhesive layer comprises at least one thermoplastic elastomer; at least one liquid rubber; at least one polyisobutylene; and at least one oil.
